(19) United States
(12) Patent Application Publication
(10) Pub. No.: US 2008/0262371 A1
(43) Pub. Date: Oct. 23, 2008

(54) Method for Adaptive Complex Wavelet Based Filtering of Eeg Signals

(76) Inventor: Elvir Causevic, New York, NY (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/575,447

(22) PCT Filed: Sep. 16, 2005
(86) PCT No.: PCT/US2005/033147

§ 371 Date: May 1, 2008

Related U.S. Application Data
(60) Provisional application No. 60/610,637, filed on Sep. 16, 2004.

(51) Int. Cl. A61B 5/0476

(52) U.S. Cl. 600/544

(57) ABSTRACT

A method for adaptive filtering of EEG signals in the wavelet domain using a nearly shift-invariant complex wavelet transform. EEG signal data is segmented into a set of K “trials” or “light averages” of M-frames of data each. These trials are overlapped by a number of frames P, where P<M. A dual-tree complex wavelet transform is computed for each light average K of EEG signal data. Next, the phase variance of each resulting normalized wavelet coefficient is computed, and the magnitude of each wavelet coefficient is selectively scaled according to the phase variance of the coefficients. The resulting wavelet coefficients are then utilized to reconstruct the ABR signal extracted from the EEG data.

### TECHNICAL FIELD

The present invention relates generally to the extraction or denoising of auditory brainstem responses (ABR) from an electroencephalogram (EEG) signal, and in particular, to a method for adaptive filtering of EEG signals in the wavelet domain using a nearly shift-invariant complex wavelet transform.

### BACKGROUND ART

Auditory evoked potential (AEP) signals are transient electrical biosignals produced by various regions of the human brain in response to auditory stimuli, such as a repetition of “clicks”. These signals are traditionally categorized into three groups. The first group is commonly referred to as the auditory brainstem response (ABR), and occurs during the first 11 ms following the stimulus. The second group is the mid-latency cortical response (MLR), also known as the mid-latency evoked potential (ML-EP), which is typically confined to the next 70 ms. The final group is the slow cortical response, which begins to occur at about 80 ms following the stimulus.

In a human subject with normal auditory response, the AEP signals have a waveform morphology which typically exhibits five waves (peaks) identified as I, II, III, IV, an V in the 1.5 ms to 7 ms interval initially following the introduction of the auditory stimulus. Specific deviations from a “normal” morphology can be mapped to specific auditory dysfunctions, neurological, or psychiatric disorders in the human patient. Hence, the AEP signals are of significant interest for clinical diagnostic purposes.

Traditionally, methods used in clinical practice almost always rely on trained experts who visually identify the AEP waveform components (usually peaks), and then compute features of interest, such as inter-peak latency I-V, from the ABR traces. In order to implement a fully-automated extraction of these inter-peak latencies and other features for the purpose of machine-made diagnostics, it would be advantageous to provide for “optimal” extraction and reconstruction of the ABR waveform components from a measured EEG signal.

However, auditory evoked potential signals are typically one order of magnitude smaller than the EEG signals, and are therefore not directly visible from a raw EEG signal trace. Conventional methods for the extraction of auditory evoked potential signals from the EEG fundamentally rely upon bandpass filtering of the EEG signal, followed by an averaging of a large number of frames of EEG signal data, all of which are synchronized to the beginning of the auditory stimulus.

For ABR signals, different filter passbands have been used, with high-pass cutoff frequencies in the range of 30-300 Hz, and lowpass cutoff frequencies typically between 1500-3000 Hz. However, it is know that selecting a high-pass frequency of 100 Hz or more, which is commonly used in ABR analysis, may distort or obscure the slow negative wave in the 10 ms region.

In an alternative method, denoised “light average” ABR signals having a higher signal-to-noise (SNR) ratio than those obtained using bandpass filtering and averaging techniques may be obtained by processing linear averages of EEG signal frames in the Fourier domain. The EEG signal data is initially segmented into a set of K “trials” or “light averages” of M-frames of data each. These trials are overlapped by a number of frames P, where P<M. Then, for each trial, a spectral analysis is performed using an L-point Fast Fourier Transform (FFT), and the phase variance across the trials for each normalized complex spectral component is computed. A low phase variance for any given spectral component indicates that the given component is likely to belong to the phase-locked, repeatable auditory evoked potential, whereas a high phase variance indicates that the given component is likely to be due to random noise present in the EEG signal data.

Each available EEG channel is then analyzed to identify all frequencies within a minimum frequency range having a phase variance below a predetermined threshold. A variance threshold parameter Tₙ is initialized to zero and is linearly increased until the cumulative range of frequencies for which phase variance is lower than Tₙ achieves the minimum frequency range Fₘᵢₙ or Tₙ hits a predetermined maximum value Tₘₐₓ. This operation is performed independently on each available EEG channel, and the frequencies selected by the algorithm are restricted to lie win the pass-band of the bandpass filter used for preprocessing.

The desired ABR signal is then reconstructed by taking the Inverse Fast Fourier Transform (IFFT) of these selected frequencies for each EEG channel. This type of filtering is adaptive to the EEG signal, since the EEG signal itself determines the characteristics of the filter.

An additional signal processing technique, commonly known as the discrete wavelet transform (DWT) has been shown to be useful for a wide range of signal processing applications, including signal compression, digital image denoising, and video denoising. While the Fourier transform is known to produce a uniform tiling of the time-frequency plane, with Fourier components that are well-localized in frequency, but not in time, the discrete wavelet transform provides wavelet coefficients which are simultaneously localized in time and frequency. Dyadic wavelet analysis corresponds to tiling the time-frequency plane with “octave” frequency bands. In the one-dimensional case, the DWT implements a filterbank made of bandpass filters whose passbands are [f_{N}/2, f_{N}], [f_{N}/4, f_{N}/2], [f_{N}/8, f_{N}/4], etc., where f_{N} indicates the Nyquist frequency, i.e. one half of the sampling frequency.

Wavelet transforms have been successfully used for denoising as long as the SNR is moderate to high, i.e., above 10 dB. However, when the desired signal is buried in high energy noise, i.e. with and SNR of less than 0 dB, as is often the case with ABR signals contained in a high-energy EEG signal, it has been shown that conventional wavelet denoising fails. An additional drawback of classical DWT is that it is not shift-invariant in most practical forms. One exception is the undecimated form of the dyadic wavelet decomposition tree, however the computational complexity and high redundancy of this form renders it unattractive for many signal processing applications.

For forms of the wavelet transform which are not shift-invariant, the energy distribution between wavelet subbands is sensitive to a small time shift of the input signal. While this is of little importance for signal compression applications, it had been suggested that this lack of shift invariance might be the reason why discrete wavelet transforms are not commonly employed in signal analysis techniques.

While the bandpass filtering and averaging techniques, and the Fast Fourier Transform analysis techniques for extracting ABR signal data from EEG signals may prove adequate in some diagnostic procedures, it would be advantageous to provide a nearly shift-invariant wavelet transform method for extracting ABR signal data from EEG signals with a higher signal-to-noise ratio, providing greater ABR signal data resolution, and allowing for more precise analysis and evaluation.

### SUMMARY OF THE INVENTION

Briefly stated, the present invention provides a method for adaptive filtering of EEG signals in the wavelet domain using a nearly shift-invariant complex wavelet transform. The EEG signal data is initially segmented into a set of K “trials” or “light averages” of M-frames of data each. These trials are overlapped by a number of frames P, where P<M. A dual-tree complex wavelet transform is computed for each light average of EEG signal data. Next, the phase variance of each resulting normalized wavelet coefficient is computed, and the magnitude of each wavelet coefficient is selectively scaled according to the phase variance of the coefficients. The resulting wavelet coefficients are then utilized to reconstruct the ABR signal extracted from the EEG data.

The foregoing and other objects, features, and advantages of the invention as well as presently preferred embodiments thereof will become more apparent from the reading of the following description in connection with the accompanying drawings.

### BRIEF DESCRIPTION OF THE DRAWINGS

In the accompanying drawings which form part of the specification:

FIG. 1 illustrates four levels of a complex wavelet tree for a real one dimensional input signal;

FIG. 2 illustrates a dual-tree complex wavelet transform comprising two trees of real filters a and b which produce the real and imaginary parts of the complex coefficients;

FIG. 3 is a graphical representation of the behavior of a scaling parameter as a function of normalized phase variance for two values of Tₘₐₓ;

FIG. 4 is representative of an averaged ABR response taken over an analysis epoch of 15 ms;

FIG. 5 is representative of an averaged ABR response taken over an analysis epoch of 12 ms;

FIG. 6 is an exemplary graph of comparative results of extracted signal quality as a function of average length for a first data sample; and

FIG. 7 is an exemplary graph of comparative results of extracted signal quality as a function of average length for a second data sample.

Corresponding reference numerals indicate corresponding parts throughout the several figures of the drawings.

### BEST MODES FOR CARRYING OUT THE INVENTION

The following detailed description illustrates the invention by way of example and not by way of limitation. The description clearly enables one skilled in the art to make and use the invention, describes several embodiments, adaptations, variations, alternatives, and uses of the invention, including what is presently believed to be the best mode of carrying out the invention.

The Complex Wavelet Transform (CWT) overcomes the shift-invariance deficiencies of the classing discrete wavelet transform, and has been successfully utilized for video image denoising applications. A CWT is based on a structure of low-pass filters and high-pass filters, each having complex coefficients to generate complex output samples. FIG. 1 illustrates four levels of a complex wavelet tree for a real one dimensional input signal x. The real and imaginary parts (r and j) of the inputs and outputs are shown separately. The energy of each CWT band is approximately constant at all levels, and is shift invariant. Unlike real wavelet transforms, the complex wavelet transform preserves the notions of phase and amplitude of the transform coefficients. Complex filters may be designed such that the magnitudes of the step responses vary slowly with input shift, and that only the phases vary rapidly. Variations in the phases of the complex wavelets are approximately linear with input shifts, thus, based on measurement of phase shifts, efficient displacement estimation is possible and interpolation between consecutive complex samples can be relatively simple and accurate.

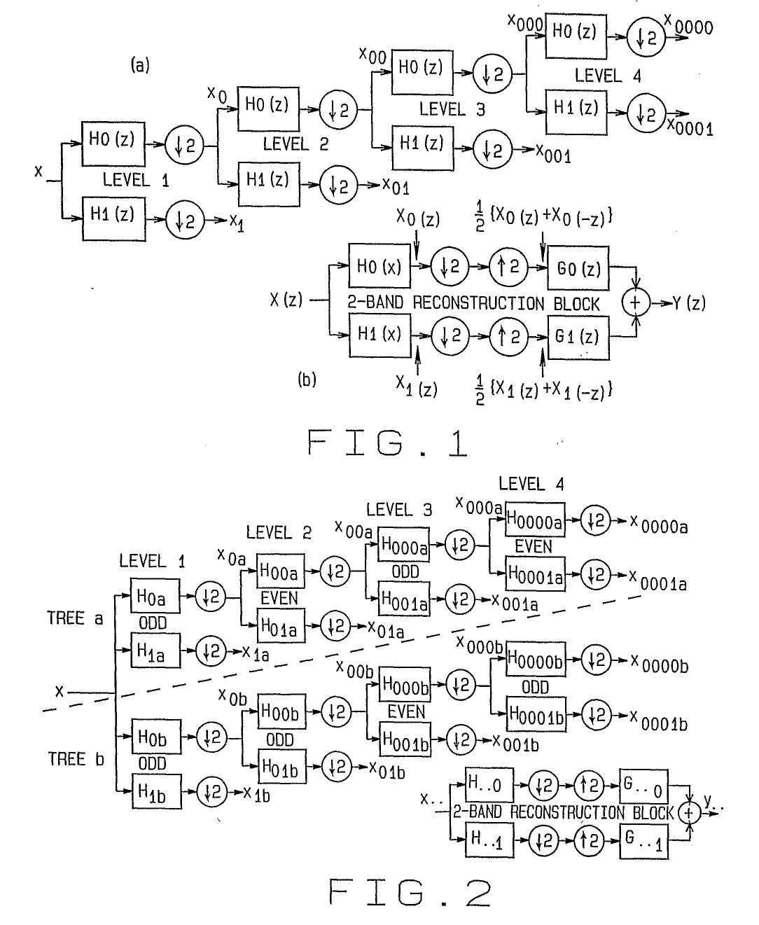

The method of the present invention utilizes a specific type of CWT referred to as a Dual-Tree Complex Wavelet Transform (DCWT), such as shown in FIG. 2, for an invertible transform in an adaptive filtering method similar to that used with conventional Fast Fourier Transforms. The complex transform coefficients of the DCWT have a magnitude and a phase, as is the case with the FFT, however, wavelet coefficients are well localized in the time-frequency plane unlike Fourier components which are only localized in frequency. Hence, setting the amplitude of a wavelet coefficient to zero will only affect a localized region in the time-domain, whereas the equivalent operation in the FFT domain affects the signal over the entire frame.

Preferably, the transform size denoted by L is selected to be 512, with eight decomposition levels or scales, such that the lowest-resolution subband consists of two coefficients.

After the Complex wavelet transform of each light average or “trial” K is computed, the phase variance of each normalized wavelet coefficient w_{i,jk} is computed according to:

$${F}_{ij} = ( \frac{1}{K} ) {\sum }_{k = 1}^{K} {\vert {w}_{ijk} - {w}_{ij} \vert}^{2}$$

where wᵢⱼ is the normalized spectral component calculated according to:

$${w}_{ijk} = \frac{{W}_{ijk}}{\vert {W}_{ijk} \vert}$$

where Wᵢⱼₖ is the i^{th} complex spectral component at wavelet scale j of the k^{th} trial, and

where wᵢⱼ is the mean normalized component calculated according to:

$${w}_{ij} = ( \frac{1}{K} ) {\sum }_{k = 1}^{K} {w}_{ijk}$$

The magnitude of each wavelet coefficient w_{i,j} is selectively scaled according to the phase variance of the coefficients at this location across the trials. Preferably, this scaling has the form:

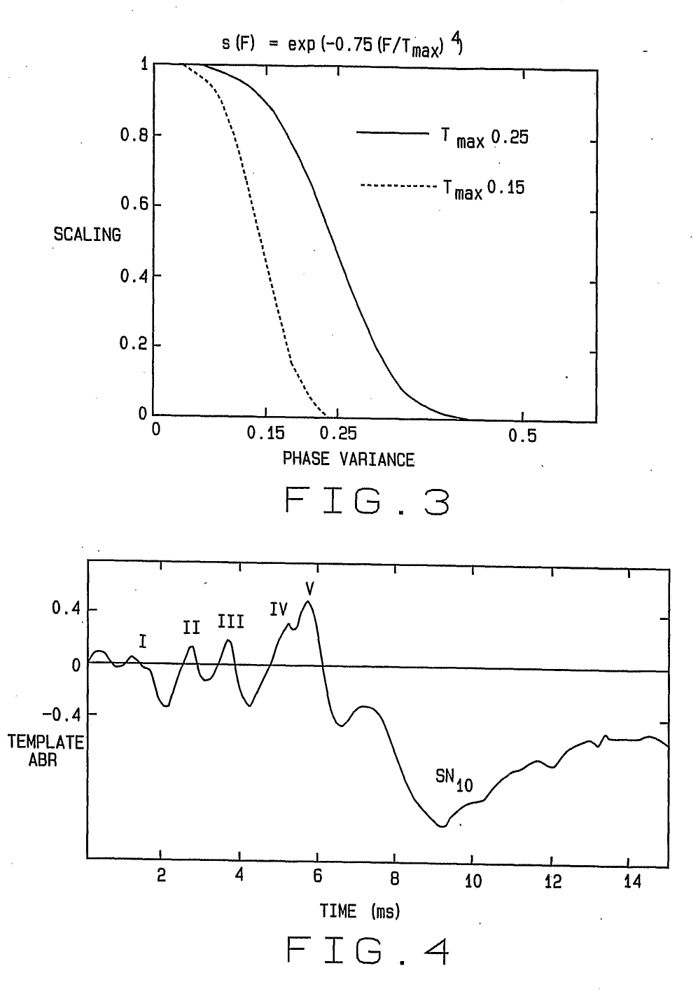

wᵢⱼ=α_{i,j}·A_{i,j}e^{jθ}^{_{i,j}}

where A_{i,j} and θ_{i,j} are respectively the magnitude and phase of the unprocessed complex i^{th} wavelet coefficient at the j^{th} scale, and where:

$${α}_{i , j} = exp ( - 0.75 \cdot {( \frac{{F}_{ij}}{{T}_{max}} )}^{4} )$$

where Fᵢⱼ is the phase variance of coefficient w_{i,j} across trials, and the parameter Tₘₐₓ is a decreasing function of the length of the short-term average used. The behavior of this scaling parameter as a function of normalized phase variances is shown in FIG. 3.

In an alternative embodiment, a “hard threshold” of the form: α_{i,j}=1 if Fᵢⱼ<Tₘₐₓ; α_{i,j}=0 is utilized in place of the scaling parameter.

The performance of the preferred method of the present invention for the denoising of auditory brainstem evoked potentials from an EEG signal is compared with conventional denoising methods below. Throughout this section, the step of bandpass filtering is denoted “BP”, the conventional linear averaging step is denoted “AVG”, the conventional adaptive filtering in the Fourier domain is denoted “AFF”, and the preferred method of the present invention for adaptive filtering in the complex wavelet domain is denoted “AFW”.

A mathematical model of digital EEG which produced signals at seven lead (electrode) locations arbitrarily referred to as Fp1, Fp2, F3, F4, F7, F8, and Fz was employed to permit objective comparison of the performance of the different algorithms. Each EEG signal has a power spectrum which approximates that of an actual EEG, i.e. which is proportional to 1/f, where f is the frequency in Hz, over a fairly wide frequency range above 30 Hz. A sampling frequency of 10 kHz was employed, sufficient to extract ABR signals since the power spectral estimates of ABR signals show little energy at frequencies above 15 kHz. Ideal models of typical averaged ABR responses taken over an analysis epoch of either 15 ms or 12 ms were employed. These models, referred to as Sample 1 and Sample 2, are shown in FIGS. 4 and 5, where peaks I-V are labeled. A final simulated EEG containing the embedded ideal models was obtained by adding the ideal model signals to each consecutive epoch of the EEG, thereby producing a signal E[n]=S[n]+N[n], where N[n] represents the biological noise contributed by the EEG.

The signal-to-noise ratio (SNR) is a convenient measure of reconstructed signal quality. Where a given signal extraction method produces an estimate S̅[n], the measure of distortion provided by the SNR, measured in dB, is given by:

SNR( S̅,S)=20 log₁₀{square root over (var(S)/var(S− S̅\sqrt{var(S)/var(S− S̅)}

where var(S) indicates the variance (or mean-square power) of S.

It is well known that the SNR (in dB) of the conventional linear averaging estimator is given by:

$$\overline{S} [ n ] = ( \frac{1}{N} ) {\sum }_{i = 1}^{N} {E}_{i} [ n ]$$

where Ei denotes the i^{th} EEG frame, and that the SNR increased by approximately 3 dB for every doubling of the length of the average N.

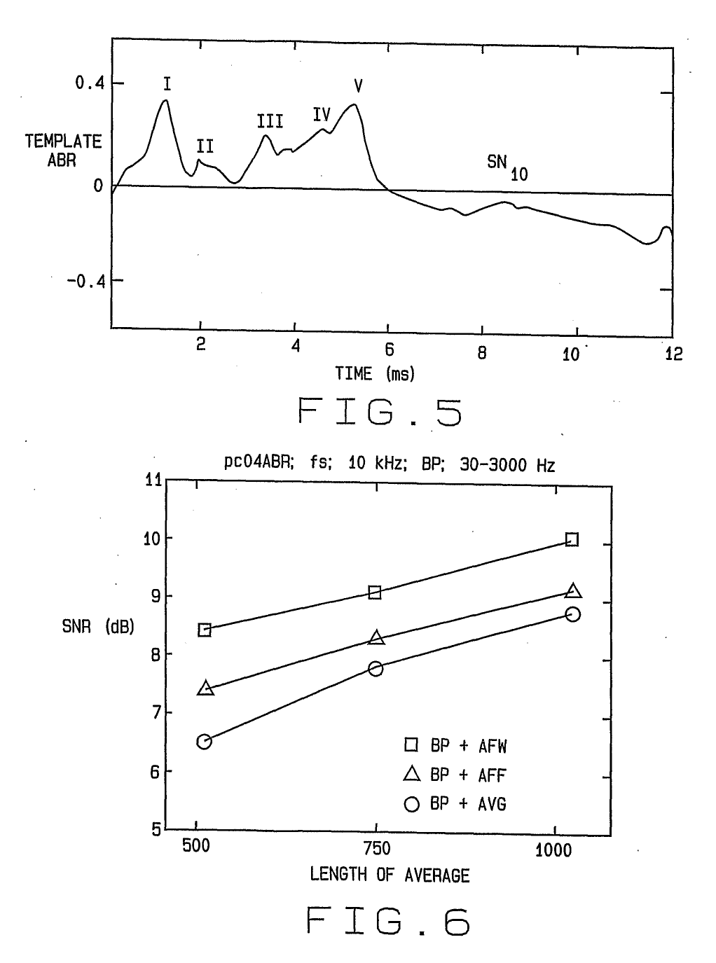

FIG. 6 and the following table illustrates a comparison of the results of extracted signal quality (in dB) for both of the conventional denoising methods, as well as for the preferred method of the present invention, using Sample 1 and three different lengths of the light averages (parameter M).

| | 512 | 750 | 1024 |
|---|---|---|---|
| BP + AVG | 6.5 (3.2) | 7.8 (3.6) | 8.8 (3.9) |
| BP + AFF | 7.4 (3.5) | 8.3 (3.7) | 9.2 (3.9) |
| | K = 8 | K = 8 | K = 8 |
| | Fmax = 950 Hz | Fmax = 950 Hz | Fmax = 950 Hz |
| | Tmax = 0.65 | Tmax = 0.65 | Tmax = 0.65 |
| | P = 256; L = 512 | P = 494; L = 512 | P = 768; L = 512 |
| BP + AFW | 8.4 (3.2) | 9.1 (3.4) | 10.1 (3.8) |
| | K = 8 | K = 8 | K = 8 |
| | Tmax = 0.25 | Tmax = 0.25 | Tmax = 0.25 |
| | P = 256; L = 512 | P = 494; L = 512 | P = 768; L = 512 |

(SNR values in dB are given as average (std); Sample rate=10 kHz, band pass filter: 30-3000 Hz.)

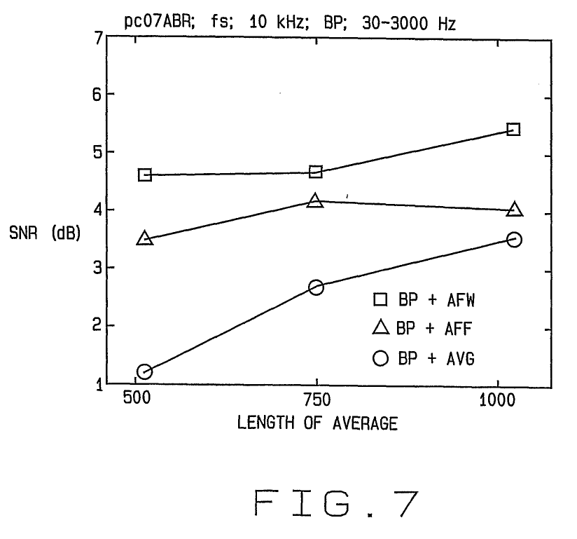

FIG. 7 and the following table illustrates a comparison of the results of extracted signal quality (in dB) for both of the conventional denoising methods, as well as for the preferred method of the present invention, using Sample 2 and three different lengths of the light averages (parameter) M.

| | 512 | 750 | 1024 |
|---|---|---|---|
| BP + AVG | 1.2 (3.1) | 2.7 (3.4) | 3.6 (3.5) |
| BP + AFF | 3.5 (2.9) | 4.2 (3.0) | 4.1 (3.0) |
| | K = 8 | K = 8 | K = 8 |
| | Fmax = 950 Hz | Fmax = 950 Hz | Fmax = 950 Hz |
| | Tmax = 0.65 | Tmax = 0.65 | Tmax = 0.65 |
| | P = 256; L = 512 | P = 494; L = 512 | P = 768; L = 512 |
| BP + AFW | 4.6 (2.8) | 4.7 (2.8) | 5.5 (2.9) |
| | K = 8 | K = 8 | K = 8 |
| | Tmax = 0.25 | Tmax = 0.25 | Tmax = 0.25 |
| | P = 256; L = 512 | P = 494; L = 512 | P = 768; L = 512 |

As is shown above, the wavelet-based method of the present invention outperforms traditional bandpass filtering followed by linear averaging, as well as conventional Fast Fourier Transform-based denoising algorithms.

The present invention can be embodied in part in the form of computer-implemented processes and apparatuses for practicing those processes. The present invention can also be embodied in part in the form of computer program code containing instructions embodied in tangible media, such as floppy diskettes, CD-ROMs, hard drives, or an other computer readable storage medium, wherein, when the computer program code is loaded into, and executed by, an electronic device such as a computer, micro-processor or logic circuit, the device becomes an apparatus for practicing the invention.

The present invention can also be embodied in part in the form of computer program code, for example, whether stored in a storage medium, loaded into and/or executed by a computer, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented in a general-purpose microprocessor, the computer program code segments configure the microprocessor to create specific logic circuits.

In view of the above, it will be seen that the several objects of the invention are achieved and other advantageous results are obtained. As various changes could be made in the above constructions without departing from the scope of the invention, it is intended that all matter contained in the above description or shown in the accompanying drawings shall be interpreted as illustrative and not in a limiting sense.

1. A method for adaptive filtering of EEG signal data to extract at least one evoked potential response, comprising:
segmenting the EEG signal data into a plurality of sets, each set including a plurality of frames of data;
overlapping each of said plurality of sets by a predetermined number of data frames;
computing a complex wavelet transform for each of said sets to identify associated normalized wavelet coefficients;
computing a phase variance of each associated normalized wavelet coefficient;
selectively scaling a magnitude of each associated normalized wavelet coefficient; and
reconstructing the at least one evoked potential response from said selectively scaled wavelet coefficients.

2. The method of claim 1 where said at least one evoked potential response is an auditory brainstem response.

3. The method of claim 1 where said step of selectively scaling a magnitude of each associated normalized wavelet coefficient is responsive to said phase variance.

4. The method of claim 1 wherein said predetermined number of data frames in said overlapping step is less than said plurality of frames of data in each set.

5. The method of claim 1 wherein said phase variance is computed from:
$${F}_{ij} = ( \frac{1}{K} ) {\sum }_{k = 1}^{K} {\vert {w}_{ijk} - {w}_{ij} \vert}^{2}$$
where wᵢₖⱼ is the normalized spectral component calculated according to:
$${w}_{ij} = \frac{{W}_{ijk}}{\vert {W}_{ijk} \vert}$$
where Wᵢⱼₖ is the i^{th} complex wavelet coefficient at wavelet scale j of the k^{th} trial, and
where wᵢⱼ is the mean normalized component calculated according to:
$${w}_{ij} = ( \frac{1}{K} ) {\sum }_{k = 1}^{K} {w}_{ijk} .$$

6. The method of claim 1 wherein said step of computing a complex wavelet transform includes computing a dual-tree complex wavelet transform for each of said sets to identify associated normalized wavelet coefficients.

7. The method of claim 1 wherein said step of scaling said magnitude of each associated normalized wavelet coefficient w_{i,j} includes computing:
wᵢⱼ=α_{i,j}·A_{i,j}e^{jθ}^{_{i,j}}
where A_{i,j} and θ_{i,j} are respectively the magnitude and phase of the unprocessed complex i^{th} wavelet coefficient at the j^{th} scale; and
where:
$${α}_{i , j} = exp ( - 0.75 \cdot {( \frac{{F}_{ij}}{{T}_{max}} )}^{4} )$$
where Fᵢⱼ is the phase variance of coefficient w_{i,j} across said sets, and the parameter Tₘₐₓ is a decreasing function.

8. The method of claim 1 wherein said step of scaling said magnitude of each associated normalized wavelet coefficient w_{i,j} includes computing:
wᵢⱼ=α_{i,j}·A_{i,j}e^{jθ}^{_{i,j}}
where A_{i,j} and θ_{i,j} are respectively the magnitude and phase of the unprocessed complex i^{th} wavelet coefficient at the j^{th} scale; and
where:
α_{i,j}1 if FᵢⱼTₘₐₓ; α_{i,j}=0
where Fᵢⱼ is the phase variance of coefficient w_{i,j} across said sets, and the parameter Tₘₐₓ is a decreasing function.
